(19) United States
(12) Patent Application Publication
(10) Pub. No.: US 2001/0053753 A1
(43) Pub. Date: Dec. 20, 2001

(54) STABLE CLEANSING COMPOSITIONS CONTAINING ACTIVE AGENTS

(76) Inventor: CAROLYN ENGEKHART, GARFIELD, NJ (US)

Correspondence Address: FINNEGAN HENDERSON FARABOW, GARRETT & DUNNER LLP, 1300 I STREET N W, WASHINGTON, DC, 200023315

(21) Appl. No.: 09/371,794

(22) Filed: Aug. 10, 1999

(51) Int. Cl. C11D 1/00; A61K 7/50; C11D 17/00; A61K 7/48; C11D 17/08

(52) U.S. Cl. 510/130; 510/159; 510/417; 510/421; 510/424; 510/472

(57) ABSTRACT

Stable cleansing compositions containing an active agent and a surfactant and further containing ethoxylated solubilizing agents, and a process for stabilizing such compositions.

This application claims the benefit of U.S. Provisional Application No. 60/096,643, filed Aug. 14, 1998.

The present invention relates to stable personal cleansing compositions comprising: at least one stabilizing agent chosen from specific ethoxylated solubilizers, particularly polyethylene glycol (“PEG”) ethers of fatty alcohols; at least one active agent, particularly a lipophilic compound; and at least one surfactant. The present invention also relates to a process for stabilizing cleansing compositions containing at least one active agent, particularly at least one lipophilic compound.

Personal cleansing compositions are useful for maintaining good skin hygiene. In so doing, personal cleansing compositions should be able to cleanse the skin of excess sebum, make-up residues, and particularly compositions which have waterproof or non-transfer properties. Conventional personal cleansing compositions use soaps based on alkali salts of fatty acids, synthetic surfactants, or mixtures of soaps and synthetic surfactants. Typically, these compositions are in the form of solid soap bars. These conventional compositions, however, have several disadvantages. In particular, conventional compositions can be harsh, removing the hydro-lipidic film of the skin, leaving the skin extremely dry. Moreover, these soap bar compositions can exhibit poor rinsability, leaving a film on the skin after rinsing. Cleansing compositions based only on synthetic surfactants, i.e., without soaps, can have problems with their foam. Often the foam obtained with these compositions is abundant but does not have the fine and dense texture offered by soaps.

Personal cleansing compositions can also act as delivery systems for active agents. “Active agents,” in accordance with the present invention, include compounds typically identified as such by those of ordinary skill in the art, for example moisturizers, emollients, and uv filters, as well as fragrances. However, certain active agents can disrupt the stability of conventional cleansing compositions. Lipophilic compounds, and fragrances in particular, especially at higher concentrations, can disrupt the stability of cleansing compositions and often suppress foam.

It is therefore an object of the present invention to provide a personal cleansing composition which contains at least one active agent, particularly at least one lipophilic agent, yet exhibits a stable viscosity profile.

It is another object of the present invention to provide a personal cleansing composition which can be fragranced yet exhibits a stable viscosity profile.

It is a further object of the present invention to provide thickened, fragranced, cleansing compositions with good foaming and conditioning properties.

It is yet another object of the present invention to provide a process for stabilizing personal cleansing compositions containing at least one active agent, particularly at least one lipophilic active agent.

Accordingly, the present invention relates to stable personal cleansing compositions comprising at least one stabilizing agent chosen from ethoxylated solubilizing agents, at least one active agent, and at least one surfactant. These compositions can further comprise water.

The present invention also relates to a process for stabilizing personal cleansing compositions comprising at least one active agent and at least one surfactant by including therein at least one stabilizing agent chosen from ethoxylated solubilizing agents.

Thus, the subject of the present invention is a personal cleansing composition containing at least one active agent and at least one surfactant, and further comprising at least one stabilizing agent, wherein the viscosity profile of the composition remains relatively constant, i.e., the composition is stable. Active agents useful in the present invention include, but are not limited to, lipophilic compounds such as retinol or its derivatives, vitamin E or its derivatives, bisabolol, oils, particularly vegetable oils, and fragrances. More preferably, the active agent is a fragrance. The surfactant used in the present invention is preferably an anionic surfactant. The stabilizing agent is preferably chosen from ethoxylated solubilizing agents. More preferably, the stabilizing agent is chosen from polyethylene glycol ethers of fatty alcohols. The inventive composition can have conditioning properties and foaming properties as well.

A “stable” composition in accordance with the present invention is either a composition whose viscosity does not change more than 20% from its initial value within 8 weeks at 45° C. or a composition whose initial viscosity is at a level useful in a cleansing composition, e.g., not lower than about 5000 cps. “Initial viscosity” is the viscosity 24 hours after manufacture of a cleansing composition. The stability of a compound can be a predictor of shelf life. For example, a compound whose viscosity does not change more than 20% within 8 weeks at 45° C. might have a shelf life of about two years at room temperature.

The present composition thus has the stability characteristics discussed above and contains at least one active agent, at least one surfactant, and at least one stabilizing agent chosen from specific solubilizers. By contrast, conventional cleansing compositions that do not contain solubilizers can exhibit a change in viscosity of greater than 20% after 8 weeks at 45° C. Furthermore, while it is generally known to use solubilizers, including ethoxylated materials, to help solubilize fragrances, some solubilizers can disrupt the viscosity of cleansing compositions, causing the initial viscosity to be too low to achieve the objectives of the present invention, e.g., about 5000 cps or less. For example, the ethoxylated solubilizer PPG-26 Buteth-26 PEG-40 hydrogenated castor oil (a mixture sold by Wackherr under the name SOLUBILISANT LRI) disrupts the viscosity of a fragranced cleansing composition, resulting in an initial viscosity of less than about 5000 cps.

The inventor has unexpectedly discovered that specific ethoxylated solubilizing agents, particularly polyethylene glycol (PEG) ethers of fafty alcohols, can stabilize compositions containing active agents, particularly lipophilic active agents. In particular, cleansing compositions comprising these specific ethoxylated solubilizing agents, particularly cleansing compositions which also comprise a fragrance, do not exhibit the change in viscosity and do not have unacceptably low viscosities, as described above.

The PEG ethers of fatty alcohols used according to the invention may preferably be chosen from compounds having formula (I), below:

CH₃RCH₂(OCH₂CH₂)ₙOH (I)

wherein:

R is chosen from alkyl and alkylene fatty chains having from about 10 to about 18 carbons; and,

n represents an average value of from about 10 to about 30.

R therefore refers to the fatty alcohol chain and n shows the average number of moles of ethylene oxide or the degree of ethoxylation. The solubilizing properties of the compositions are related to R, n, and the degree of saturation of the fatty chain. The longer the chain making up R, the more lipophilic the compound, and the higher the n, the more hydrophilic the compound. A person of ordinary skill in the art will know how to vary the parameters to achieve a compound which is effective as a solubilizer and, similarly, that the choice of n and R depend on the compound that is being solubilized.

A person of ordinary skill will also recognize that the numbers used in the definition of n and R, above, represent preferred embodiments, and that depending on how each parameter is selected, R and n may vary outside the preferred range . Thus, the inventor has used the term “about” in defining n and R to represent values, including average values, in combination sufficient to achieve stabilization of compositions of the invention containing active agents, particularly lipophilic active agents, such as fragrances.

The preferred PEG ethers of fatty alcohols of formula (I) have an R with a chain length from 10 to 16 carbons, even more preferably 14 to 16, and an average n value from 15 to 25, even more preferably 20. Particularly preferred are CETETH-20, which contains 16 carbons in its fatty chain, i.e., including the portion of its fatty chain corresponding to R in formula (I) above as well as the two additional carbon atoms on either side of R, and an n of average value 20, or STEARETH-20, which has a 18-carbon chain (where the chain length is as defined above for CETETH-20) and an n of average value 20, both available from ICI Americas.

When the active agent is a fragrance, the fragrance is present in the inventive composition in an amount up to 5%, preferably in an amount ranging from 2 to 5%. Furthermore, the ratio of the PEG ethers of fatty alcohols to the fragrance in the inventive composition can range from about 1:4 to about 5:1, based on weight. The fragrance(s) can be chosen from conventional fragrances suitable for use in personal cleansing compositions.

The composition of the present invention can also contain one or more anionic surfactants, or one or more other surfactants. Anionic surfactants suitable for use in the inventive composition include, but are not limited to alkyl sulfates, alkyl ether sulfates, alkali metal alkylarylsulfonates, alkali metal, alkanolamines, salts of alkyl polyether carboxylic acids, salts of alkylphenyl polyether carboxylic acids, and N-acylamino acids. These anionic surfactants are preferably present in the composition in concentrations of from 8% to 25% and can provide foam properties.

Thus, a preferred formulation of the present composition contains:

a) PEG ether(s) of fatty alcohols in an amount of from 2% to 25%,

b) anionic surfactant(s) in an amount of from 8% to 25%,

c) fragrance(s) in an amount of from 2% to 5% and

d) water.

All percentages and/or ratios used herein are by weight of the total composition unless otherwise indicated.

Other surfactants may be used alone, or preferably, in combination with the anionic surfactants. For example, certain cleansing compositions, such as baby shampoos, that are intended to be particularly mild may contain amphoteric surfactants. Preferred classes of amphoteric surfactants include: N-alkylamino acids, N-alkylamine oxides, alkylimidazolines, betaines containing fatty chains, or amidobetaines derived from fatty acids, or salts of amino acids such as N-lauryl β-alanine and N-stearyl β-alanine. Preferred nonionic surfactants of the following classes may be used: alkanolamides, alkyl ethers of polyglycerols, alkyl ethers of polyethylene glycols, alkyl glucosides, copolymers of ethylene oxide and propylene oxide.

The composition according to the invention is advantageously in a thickened form, preferably a gel. Therefore, the composition preferably also comprises at least one thickener or gelling agent. The thickeners or gelling agents can be either synthetic or natural and are preferably chosen from carboxylic acid polymers, crosslinked polyacrylate polymers, polyacrylamide polymers, polysaccharides, gums, gelatins, vinyl ether/maleic anhydride copolymers, and crosslinked poly (N-vinylpyrrolidones).

The at least one thickener or gelling agent is preferably present in the composition in an amount ranging from 0.1% to 2%, more preferably from 0.25% to 1% and even more preferably from 0.4% to 0.8%. The thickener or gelling agent can be a single compound as well as a mixture of two or more compounds.

The composition of the present invention can also comprise any ingredient usually used by any person of skill in the art in the personal care area. Such ingredients can be, but are not limited to, adjuvants such as natural and synthetic polymers, colorants, preservatives, antioxidants, medicaments, moisturizers, sunscreen agents, germicides, deodorants, opacifiers, pearlizing agents, oils, waxes, thickeners for the oily phase, proteins, amino acids, healing agents, solvents, humectants, emollients, buffers, pH adjusters, chelating agents, and abrasives. A preferred embodiment includes a pearlizing agent.

A person skilled in the art will choose the other ingredients usually associated with a like composition in a way that will substantially maintain the advantages of the present invention.

Another subject of the present invention is a process for stabilizing personal cleansing compositions containing at least one active agent, by including therein specific ethoxylated solubilizing agent(s). In a preferred embodiment, PEG ethers of fatty alcohols are included in cleansing compositions comprising at least one fragrance.

The following examples are intended to illustrate the invention and are not intended to be limiting. All ingredients are named using the CTFA nomenclature, as set forth in the Seventh Edition of International Cosmetic Ingredient Dictionary and Handbook.

### Example 1 (Comparative)

The following composition was made using no solubilizer for the active agent, in this case a fragrance.

| Ingredients | % w/w |
|---|---|
| Water | q.s. |
| Acrylates/C10-30 alkyl | 0.40 |
| acrylate crosspolymer | |
| Disodium EDTA | 0.05 |
| Methylparaben | 0.20 |
| Propylparaben | 0.15 |
| Sodium laureth sulfate | 12.35 |
| Cocamidopropyl betaine | 8.60 |
| Triclosan | 0.05 |
| Glycol distearate | 2.00 |
| Cocamide MEA | 2.00 |
| Sodium hydroxide | 0.10 |
| Polyquaternium-7 | 1.25 |
| Phenoxyethanol | 0.30 |
| Corn oil | 0.10 |
| BHT | 0.07 |
| Fragrance | 4.00 |
| | 100.00 |

A first premix of sodium hydroxide and water was prepared by mixing the two ingredients with a magnetic stir bar at 25° C. until uniform.

A second premix was prepared by mixing polyquaternium-7 and water together at 25° C. using a magnetic stir bar.

A third premix of fragrance, corn oil and BHT was prepared at 35°-40° C. using a magnetic stir bar.

Water was heated to 75°-80° C. The following ingredients were mixed into the water until dissolved, while maintaining the 750-80° C. temperature, sequentially as follows:

acrylates/C10-30 alkyl acrylate crosspolymer;

disodium EDTA, methylparaben and propylparaben,

sodium laureth sulfate,

cocamidopropyl bentaine and triclosan.

Mixing continued until the dispersion appeared uniform. The mixture was cooled to 70°-75° C. and glycol distearate added and mixed in until melted and dissolved. Cocamide MEA was then mixed until dissolved and the mixture was uniform. The blend was then cooled until 65°-70° C. wherein the first premix was blended in until the mixture was uniform. After cooling until 30°-35° C., the second premix was mixed in until uniform, followed by phenoxyethanol which was also mixed in until uniform. Finally, the 30 premix was added and mixed in until the blend was uniform. The mixture was cooled to 25° C.

The viscosity of the composition was measured 24 hours after manufacture using a Brookfield RVT using spindle #5 rotating at 20 rpm for 1 minute at 25° C. The viscosity measurements are shown below in Table 1. The initial viscosity of 15450 centipoise (cps) dropped to 7650 cps after 2 weeks at 45° C. It remained fairly constant after that with a reading of 8750 cps after 8 weeks at 45° C. Thus, when no solubilizer is used, the viscosity drops about 50% in two weeks and remains at about that level.

TABLE 1

| Time | Viscosity |
|---|---|
| Initial | 15450 cps |
| After 2 weeks @ 45° C. | 7650 cps |
| After 8 weeks @ 45° C. | 8750 cps |

A similar drop in viscosity (50% drop or more) was observed after one or more freeze-thaw cycles. The freeze-thaw process was carried out by placing the composition in a chamber that cycled between a temperature of −10° C. and a temperature of 25° C. The composition was maintained at each temperature for about 12 hours, with less than 1 hour transition between the two temperatures. Thus, one cycle was 24 hours in length. After one cycle the viscosity dropped from the initial 15450 cps to 7100 cps, after 6 cycles the viscosity was 6100 cps.

TABLE 2

| Time | Thaw |
|---|---|
| (Freeze-Thaw Cycles) | (Cps) |
| Initial | 15450 |
| After 1 | 7100 |
| After 6 | 6100 |

Thus, when no solubilizer is used, the viscosity reduction is about 55%.

### Example 2 (Comparative)

| Ingredients | % w/w |
|---|---|
| Water | q.s. |
| Acrylates/C10-30 alkyl | 0.40 |
| acrylate crosspolymer | |
| Disodium EDTA | 0.05 |
| Methylparaben | 0.20 |
| Propylparaben | 0.15 |
| Sodium laureth sulfate | 12.35 |
| Cocamidopropyl betaine | 8.60 |
| Triclosan | 0.05 |
| Glycol distearate | 2.00 |
| Cocamide MEA | 2.00 |
| Sodium hydroxide | 0.10 |
| Polyquaternium-7 | 1.25 |
| Phenoxyethanol | 0.30 |
| Corn oil | 0.10 |
| BHT | 0.07 |
| PPG-26 Buteth-26 PEG-40 | 4.00 |
| hydrogenated castor oil | |
| Fragrance | 4.00 |
| TOTAL | 100.00 |

The product in this example was made according to the process outlined in Example 1, except that the solubilizer was premixed along with the fragrance, the corn oil and the BHT. The product was made with 4% PPG-26 Buteth-26 PEG-40 Hydrogenated castor oil as a solubilizer for the fragrance. The viscosity of the initial composition was too low, i.e., less than 5000 cps. Thus, the viscosity was aesthetically unacceptable to achieve the objectives of the present invention and additional stability studies were not warranted.

### Example 3 (Comparative)

| Ingredients | % w/w |
|---|---|
| Water | q.s. |
| Acrylates/C10-30 alkyl | 0.40 |
| acrylate crosspolymer | |
| Disodium EDTA | 0.05 |
| Methylparaben | 0.20 |
| Propylparaben | 0.15 |
| Sodium laureth sulfate | 12.35 |
| Cocamidopropyl betaine | 8.60 |
| Triclosan | 0.05 |
| Glycol distearate | 2.00 |
| Cocamide MEA | 2.00 |
| Sodium hydroxide | 0.10 |
| Polyquaternium-7 | 1.25 |
| Phenoxyethanol | 0.30 |
| Corn oil | 0.10 |
| BHT | 0.07 |
| Polysorbate-20 | 4.00 |
| Fragrance | 4.00 |
| TOTAL | 100.00 |

The product in this example, and in the subsequent examples, was made in the same manner as Example 2. The product in this Example 3 was made with 4% Polysorbate-20, a polyoxyethylene sorbitan laurate ester used as a solubilizer to replace the 4% PPG-26 Buteth-26 PEG-40 Hydrogenated castor oil. The viscosity was too low, i.e. less than 5000 cps, and the product was deemed aesthetically unacceptable to achieve the objectives of the present invention.

### Example 4 (Inventive)

| Ingredients | % w/w |
|---|---|
| Water | q.s. |
| Acrylates/C10-30 alkyl | 0.40 |
| acrylate crosspolymer | |
| Disodium EDTA | 0.05 |
| Methylparaben | 0.20 |
| Propylparaben | 0.15 |
| Sodium laureth sulfate | 12.35 |
| Cocamidopropyl betaine | 8.60 |
| Triclosan | 0.05 |
| Glycol distearate | 2.00 |
| Cocamide MEA | 2.00 |
| Sodium hydroxide | 0.10 |
| Polyquaternium-7 | 1.25 |
| Phenoxyethanol | 0.30 |
| Corn oil | 0.10 |
| BHT | 0.07 |
| Fragrance | 4.00 |
| STEARETH-20 | 2.00 |
| | 100.00 |

The product in this example was made by replacing the PPG-26 Buteth-26 PEG-40 hydrogenated castor oil of Example 2 with STEARETH-20. The viscosity was measured on a Brookfield RVT viscometer using spindle #6 under the same conditions as those in Example 1. The viscosity measurements are shown below in Table 3. This initial viscosity of 7160 cps dropped only to 5820 cps (a 18.7% drop) after 2 weeks at 45° C. The viscosity showed a slight improvement over time with a reading of 6740 cps (a 5.9% reduction in viscosity) after 8 weeks at 45° C. The freeze-thaw stability measurements are shown below in Table 4. The freeze-thaw stability was also improved showing a reduction of the viscosity to 6360 cps (11.2%) reduction after 1 cycle and a viscosity of 7120 cps (a reduction of 0.6%) after 6 cycles. This shows that STEARETH-20 did not negatively affect the viscosity as compared to the PPG-26 Buteth-26 PEG-40 hydrogenated castor oil of Example 2. In particular, the composition comprising STEARETH-20 exhibited an acceptable initial viscosity and a relatively constant viscosity profile, whereas the composition containing the PPG-26 Buteth-26 PEG-40 hydrogenated castor oil had an initial viscosity that was too low.

TABLE 1

| Time | Viscosity |
|---|---|
| (Weeks) | (Cps) |
| Initial | 7160 |
| After 2 weeks at 45° C. | 5820 |
| After 8 weeks at 45° C. | 6740 |

TABLE 2

| Time | Viscosity |
|---|---|
| (Freeze-Thaw Cycles) | (Cps) |
| After 1 | 6360 |
| After 6 | 7120 |

### Example 5 (Inventive)

| Ingredients | % w/w |
|---|---|
| Water | q.s. |
| Acrylates/C10-30 alkyl | 0.40 |
| acrylate crosspolymer | |
| Disodium EDTA | 0.05 |
| Methylparaben | 0.20 |
| Propylparaben | 0.15 |
| Sodium laureth sulfate | 12.35 |
| Cocamidopropyl betaine | 8.60 |
| Triclosan | 0.05 |
| Glycol distearate | 2.00 |
| Cocamide MEA | 2.00 |
| Sodium hydroxide | 0.10 |
| Polyquaternium-7 | 1.25 |
| Phenoxyethanol | 0.30 |
| Corn oil | 0.10 |
| BHT | 0.07 |
| Fragrance | 4.00 |
| CETETH-20 | 4.00 |
| | 100.00 |

The product in this example was made in the same manner as in Example 4 by replacing the STEARETH-20 with CETETH-20. The composition of Example 5 exhibited an initial viscosity comparable to the composition containing STEARETH-20. Although stability studies were not carried out, the result would be expected to be similar to those of Example 4.

What is claimed is:

1. A stable personal cleansing composition comprising:
at least one stabilizing agent chosen from ethoxylated solubilizing agents,
at least one surfactant, and
at least one active agent.

2. The composition according to claim 1, wherein said ethoxylated solubilizing agent is chosen from polyethylene glycol ethers of fafty alcohols.

3. The composition according to claim 2, further comprising at least one anionic surfactant.

4. The composition according to claim 3, wherein said at least one anionic surfactant is present in said composition in an amount ranging from 8% to 25% based on the total weight of the composition.

5. The composition according to claim 3, wherein said at least one anionic surfactant is chosen from alkyl sulfates, alkyl e ther sulfates, alkali metal alkylarylsulfonates, alkali metal alkanolamines, salts of alkyl polyether carboxylic acids, salts of alkylphenyl polyether carboxylic acids, and N-acylamino acids.

6. The composition according to claim 1, further comprising water.

7. The composition according to claim 1, wherein said at least one active agent is chosen from lipophilic compounds.

8. The composition according to claim 1, wherein said at least one active agent is a fragrance.

9. The composition according to claim 2, wherein:
said at least one polyethylene glycol ether of fatty alcohols is present in said composition in an amount ranging from 2% to 25%;
said at least one surfactant is chosen from anionic surfactants and is present in said composition in an amount ranging from 8% to 25%; and,
said at least one active agent is a fragrance and is present in said composition in an amount ranging from 2% to 5%, the percentage amounts being based on the weight of the total composition.

10. The composition according to claim 2, wherein said at least one polyethylene glycol ether of fatty alcohols contains from 10 to 30 moles of ethylene oxide and a fatty alcohol chain length from 12 to 20 carbons.

11. The composition according to claim 8, wherein said at least one stabilizing agent and said at least one fragrance are present in a ratio of from 1:4 to 5:1, wherein the ratio is a weight/weight ratio.

12. The composition according to claim 2, wherein the at least one polyethylene glycol ether of fatty alcohols is chosen from compounds of formula (1),
CH₃RCH₂(OCH₂CH₂)ₙOH (1)
wherein:
R is chosen from alkyl and alkylene fatty chains of from about 10 to about 18 carbons; and,
n has an average value of from about 10 to about 30.

13. The composition according to claim 12, wherein R is chosen from alkyl and alkylene fatty chains of from 10 to 16 carbons.

14. The composition according to claim 13, wherein n has an average value of from 15 to 25.

15. The composition according to claim 14, wherein R is chosen from alkyl and alkylene fatty chains of from 14 to 16 carbons, and n has an average value of 20.

16. The composition according to claim 8, further comprising at least one thickening agent.

17. The composition according to claim 16, wherein said at least one thickening agent is chosen from carboxylic acid polymers, crosslinked polyacrylate polymers, polyacrylamide polymers, polysaccharides, gums, gelatins, vinyl ether/maleic anhydride copolymers, and crosslinked poly (N-vinylpyrrolidones).

18. The composition according to claim 16, wherein said at least one thickening agent is present in said composition in an amount ranging from 0.1% to 2% based on the total weight of the composition.

19. The composition according to claim 18, wherein the amount of said at least one thickening agent ranges from 0.25% to 1%.

20. The composition according to claim 19, wherein the amount of said at least one thickening agent ranges from 0.4% to 0.8%.

21. The composition according to claim 16, further comprising at least one additional adjuvant chosen from natural and synthetic polymers, colorants, preservatives, antioxidants, medicaments, moisturizers, uv filters, germicides, deodorants, opacifiers, pearlizing agents, oils, waxes, thickeners for the oily phase, proteins, amino acids, healing agents, solvents, humectants, emollients, buffers, pH adjusters, chelating agents, abrasives, and surfactants other than anionic surfactants.

22. The composition according to claim 21, wherein said at least one adjuvant is a pearlizing agent.

23. A process for stabilizing a cleansing composition, comprising:
including an effective amount of at least one stabilizing agent chosen from ethoxylated solubilizing agents in a cleansing composition containing at least one at least one surfactant and at least one active agent.

24. A process according to claim 23, wherein said at least one ethoxylated solubilizing agent is chosen from polyethylene glycol ethers of fafty alcohols.

25. A process according to claim 24, wherein said at least one active agent is a fragrance.

26. A personal cleansing composition comprising:
at least one polyethylene glycol ether of a fatty alcohol, at least one anionic sufactant, and at least one fragrance.
